(19) United States
(12) Patent Application Publication
(10) Pub. No.: US 2007/0066594 A1
(43) Pub. Date: Mar. 22, 2007

(54) Crystalline forms fenoldopam mesylate

(76) Inventors: Peter Lindsay MacDonald, Gentilino (CH); Ettore Bigatti, Balerna (CH); Pierluigi Rossetto, Balerna (CH); Judith Aronhime, Rehovot (IL); Sigalit Levi, Modi'in (IL)

Correspondence Address: KENYON & KENYON LLP, ONE BROADWAY, NEW YORK, NY, 10004, US

(21) Appl. No.: 11/505,229

(22) Filed: Aug. 15, 2006

Related U.S. Application Data
(60) Provisional application No. 60/708,739, filed on Aug. 15, 2005.

(51) Int. Cl. A61K 31/55; C07D 223/16

(52) U.S. Cl. 514/217.02; 540/595

(57) ABSTRACT

The invention provides new crystalline forms of fenoldopam mesylate, i.e. fenoldopam mesylate Type I, fenoldopam mesylate Type III, fenoldopam mesylate Type V, and fenoldopam mesylate Type VI, methods of preparing the crystalline forms, and pharmaceutical compositions comprising the fenoldopam mesylate crystalline forms of the invention.

### CROSS-REFERENCE TO RELATED APPLICATIONS

This application claims the benefit of U.S. provisional application Ser. No. 60/708,739, filed Aug. 15, 2005, incorporated herein by reference.

### FIELD OF THE INVENTION

The present invention is directed to polymorphs of fenoldopam mesylate. In particular, the present invention is directed to new crystalline forms of fenoldopam mesylate, to crystallization processes for the new crystalline forms, and to pharmaceutical compositions comprising at least one of the crystalline structures of fenoldopam mesylate.

### BACKGROUND OF THE INVENTION

Fenoldopam mesylate, 6-chloro-2,3,4,5-tetrahydro-1-(4-hydroxyphenyl)-[1H]-3-benzazepine-7,8-diol methanesulfonate, is a white to off-white powder of chemical formula

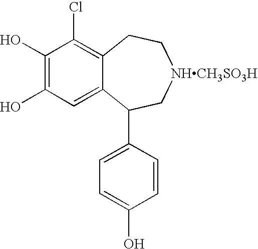

Fenoldopam mesylate injection, U.S.P., or CORLOPAM°, is a dopamine D1-like receptor agonist. The product is formulated as a solution to be diluted for intravenous infusion, and is indicated for the in-hospital short-term management of severe hypertension.

The present invention relates to the solid state physical properties of fenoldopam mesylate. These properties can be influenced by controlling the conditions under which fenoldopam mesylate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences, as it imposes an upper limit on the rate at which an orally-administered active ingredient can reach the patient's bloodstream. The rate of dissolution is also a consideration in formulating syrups, elixirs, and other liquid medicaments. The solid state form of a compound may also affect its behavior on compaction and its storage stability.

The discovery of new polymorphic forms of a pharmaceutically useful compound provides a new opportunity to improve the performance characteristics of a pharmaceutical product. It enlarges the repertoire of materials that a formulation scientist as available for designing, for example, a pharmaceutical dosage form of a drug with targeted release profile or other desired characteristic. There is a need in the art for additional crystalline forms of fenoldopam mesylate.

### SUMMARY OF THE INVENTION

The present invention is directed to crystalline forms of fenoldopam mesylate, to methods of preparing crystalline forms of fenoldopam mesylate, and to pharmaceutical compositions comprising such crystalline forms of fenoldopam mesylate.

In one embodiment, the invention is directed to a fenoldopam mesylate crystalline form characterized by data selected from the group consisting of a PXRD pattern with peaks at about 16.4°, 18.8°, 21.8°, 23.9°, and 30.8° 2θ±0.2° 2θ and a Fourier transform infrared spectroscopy (“FTIR”) spectrum with characteristic absorption bands in units of cm⁻¹ at about 1210, 1571, 1643, 3178, and 3450 cm⁻¹.

In a another embodiment, the invention is directed to a fenoldopam mesylate crystalline form characterized by data selected from the group consisting of a PXRD pattern with peaks at about 17.5°, 19.2°, 21.2°, 23.4°, and 25.3° 2θ±0.2° 2θ and a Fourier transform infrared spectroscopy (“FTIR”) spectrum with characteristic absorption bands in units of cm¹ at about 588, 1170, 1198, 1439, and 1587 cm⁻¹.

In a further embodiment, the invention is directed to a fenoldopam mesylate crystalline form characterized by data selected from the group consisting of a PXRD pattern with peaks at about 9.4°, 19.2°, 20.6°, 21.8°, and 25.3° 2θ±0.2° 2θ and a Fourier transform infrared spectroscopy (“FTIR”) spectrum with characteristic absorption bands in units of cm⁻¹ at about 1159, 1430, 1497, 1639, and 3542 cm⁻¹.

In a further embodiment, the invention is directed to a fenoldopam mesylate crystalline form characterized by data selected from the group consisting of a PXRD pattern having peaks at about 17.3°, 19.7°, 23.0°, 24.3°, and 30.0° 2θ±0.2° 2θ and a Fourier transform infrared spectroscopy (“FTIR”) spectrum with characteristic absorption bands in units of cm⁻¹ at about 559, 1259, 1579, 3168, and 3642 cm⁻¹.

In a further embodiment, the invention is directed to a process for the preparation of fenoldopam mesylate Type II, comprising providing a solution comprising isopropanol and fenoldopam mesylate; crystallizing to obtain fenoldopam mesylate; and recovering the fenoldopam mesylate Type II.

### BRIEF DESCRIPTION OF THE DRAWINGS

FIG. 1. illustrates an XRD diffractogram of fenoldopam mesylate Type I;

FIG. 2. illustrates an XRD diffractogram of fenoldopam mesylate Type II;

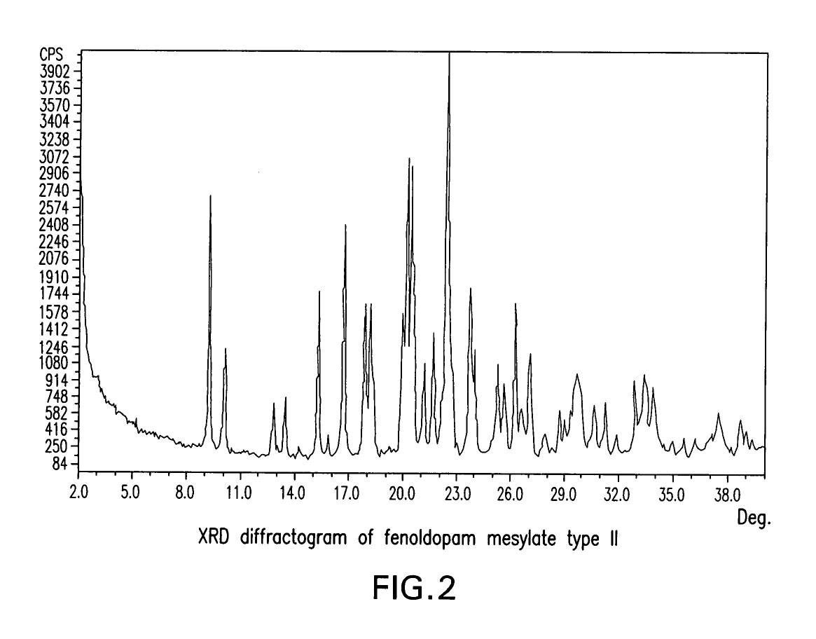

FIG. 3. illustrates an XRD diffractogram of fenoldopam mesylate Type III;

FIG. 4. illustrates an XRD diffractogram of fenoldopam mesylate Type V;

FIG. 5. illustrates an XRD diffractogram of fenoldopam mesylate Type VI;

FIG. 6. illustrates an FTIR spectra of fenoldopam mesylate Type I;

FIG. 7. illustrates an FTIR spectra of fenoldopam mesylate Type II;

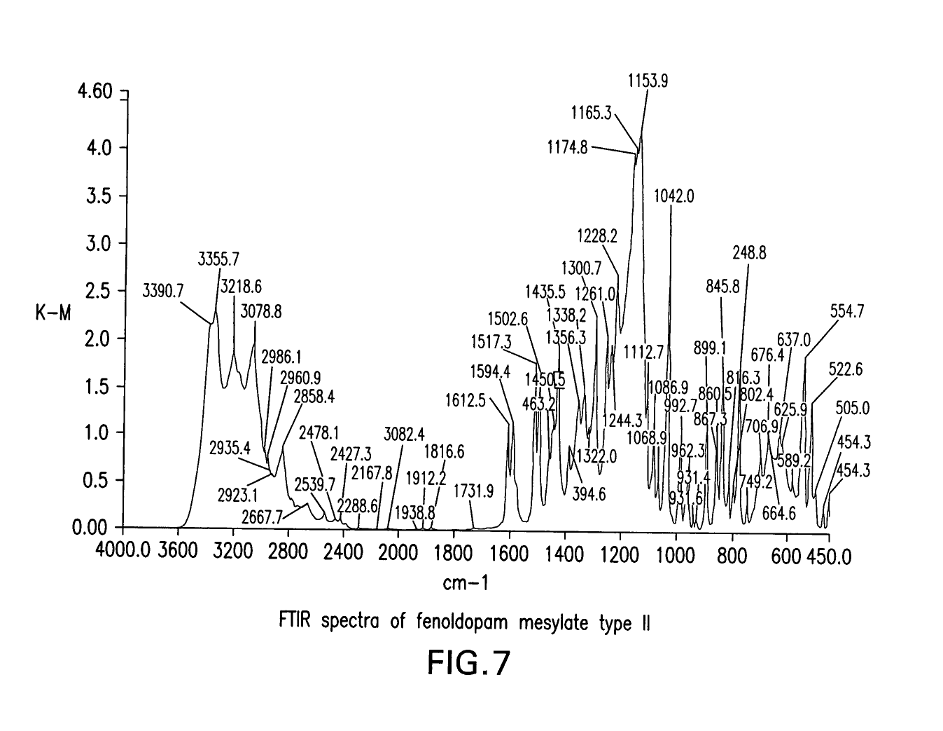

FIG. 8. illustrates an FTIR spectra of fenoldopam mesylate Type III;

FIG. 9. illustrates an FTIR spectra of fenoldopam mesylate Type V; and

FIG. 10. illustrates an FTIR spectra of fenoldopam mesylate Type VI;

### DETAILED DESCRIPTION OF THE PREFERRED EMBODIMENTS OF THE INVENTION

The present invention is directed to crystalline forms of fenoldopam mesylate, and methods for preparation thereof. Preferably, the crystalline forms of fenoldopam mesylate of the invention are preferably at least about 10 percent by weight pure, more preferably, at least about 25 percent by weight pure, and, most preferably, at least about 50 percent by weight pure. Particularly preferred crystalline forms of fenoldopam mesylate of the invention are from about 90 percent to substantially 100 percent by weight percent pure.

The USP reference standard CAT#1269458 was characterized by a powder X-ray diffraction (“XRD”) spectrum with peaks in degrees 2θ±0.2° 2θ, as well as absorption bands of an FTIR spectrum in units of cm⁻¹. The data are summarized in Table 1.

TABLE 1
Type II

| FTIR | PXRD |
|---|---|
| 1165.3 | 9.2 |
| 1228.2 | 15.3 |
| 1435.5 | 16.7 |
| 1503 | 17.9 |
| 1517 | 18.2 |
| 1594 | 20.2 |
| 1612 | 20.5 |
| 3079 | 22.4 |
| 3219 | 23.7 |
| 3356 | 26.3 |

In one embodiment, the invention is directed to a fenoldopam mesylate hydrate.

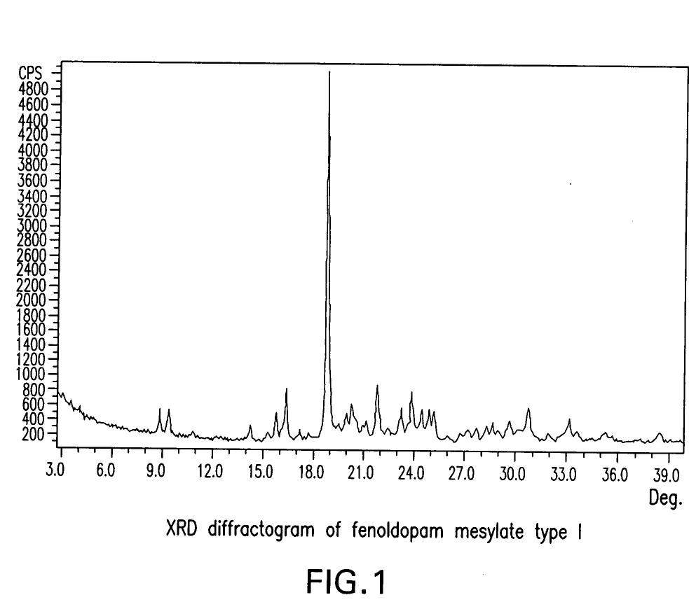

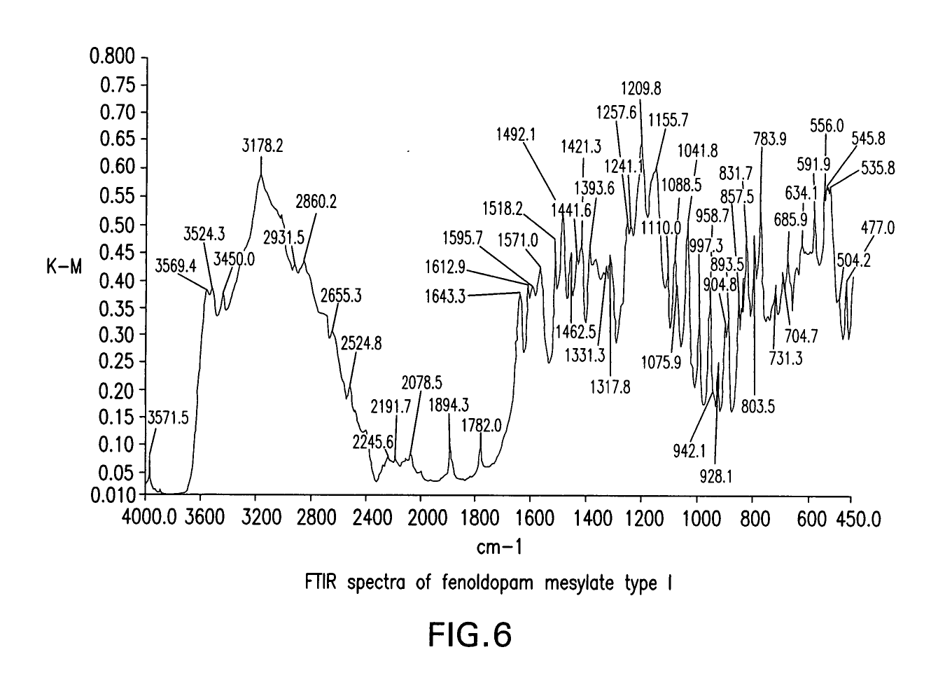

In another embodiment, the invention is directed to a fenoldopam mesylate crystalline form characterized by data selected from the group consisting of a PXRD pattern with peaks at about 16.4°, 18.8°, 21.8°, 23.9°, and 30.8° 2θ±0.2° 2θ and a Fourier transform infrared spectroscopy (“FTIR”) spectrum with characteristic absorption bands in units of cm⁻¹ at about 1210, 1571, 1643, 3178, and 3450 cm⁻¹. This fenoldopam mesylate crystalline is denominated herein as “Type I”. The Fenoldopam mesylate crystalline may be further characterized by data selected from the group consisting of a PXRD pattern with peaks at about 8.8°, 9.4°, 15.8°, 20.3°, and 23.3° 2θ±0.2° 2θ; a FTIR spectrum having absorption peaks at about 536, 546, 959, 1421, and 1442 cm⁻¹; a PXRD pattern substantially as depicted in FIG. 1; and a FTIR absorption spectrum substantially as depicted in FIG. 6. Preferably, the fenoldopam mesylate is a hydrate, having a TGA weight loss of 5.5 to about 8.0 percent by weight over a temperature range of 25° to 130° C. TGA measurements provided results similar to the water content determined by Karl Fisher, demonstrating that fenoldopam mesylate Type I is a dihydrate, wherein the theoretical water content of the dihydrate form is about 8.2 percent by weight. In addition, the crystalline form is stable to exposure to 0 to 100% relative humidity conditions, i.e., remains as the Type I dihydrate, for more than 5 days.

The present invention further provides substantially pure Fenoldopam mesylate Type I containing less than about 5%, preferably, less than about 1% of any other crystalline form of Fenoldopam mesylate, as measured by XRD, wherein any other crystalline form includes Types II, III, V, and VI.

Fenoldopam mesylate Type I may be formed by providing a solution comprising fenoldopam mesylate and water to form a solution; crystallizing fenoldopam mesylate from the solution; and recovering fenoldopam mesylate Type I.

Preferably, dissolving fenoldopam mesylate in water is by heating a combination of fenoldopam mesylate and water to a temperature of about 20° C. to about 100° C. More preferably, the heating temperature is about 60° C. to about 80° C. Preferably, dissolving fenoldopam mesylate is by addition of an alcohol, preferably methanol. Preferably, the solution is acidified using methanesulfonic acid to a pH of about 2 to about 4. Preferably, the acid is in an amount sufficient to provide the desired pH. Preferably, the solution is clarified by filtration before crystallizing. Preferably, the solution volume is reduced under vacuum at a temperature from about 50° to about 90° C. to form a suspension and initiate crystallization, especially when using an alcohol. As used herein, the term “vacuum” refers to an ambient pressure of less than about 100 mm Hg. Preferably, crystallizing is by cooling to a temperature of about 0° to about 25° C., more preferably to a temperature of about 100 to about 0° C. Preferably, recovery of the fenoldopam mesylate Type I is by any means known in the art for example, filtration, washing, and drying. Preferably, fenoldopam mesylate Type I crystals are washed with water, and dried under vacuum at a temperature of about 50° to about 90° C.

Alternatively, fenoldopam mesylate Type I may be prepared by exposing fenoldopam mesylate Type V to more than 80% relative humidity for more than about 7 days. Preferably, exposure is at room temperature. Preferably, the relative humidity is more than about 90%

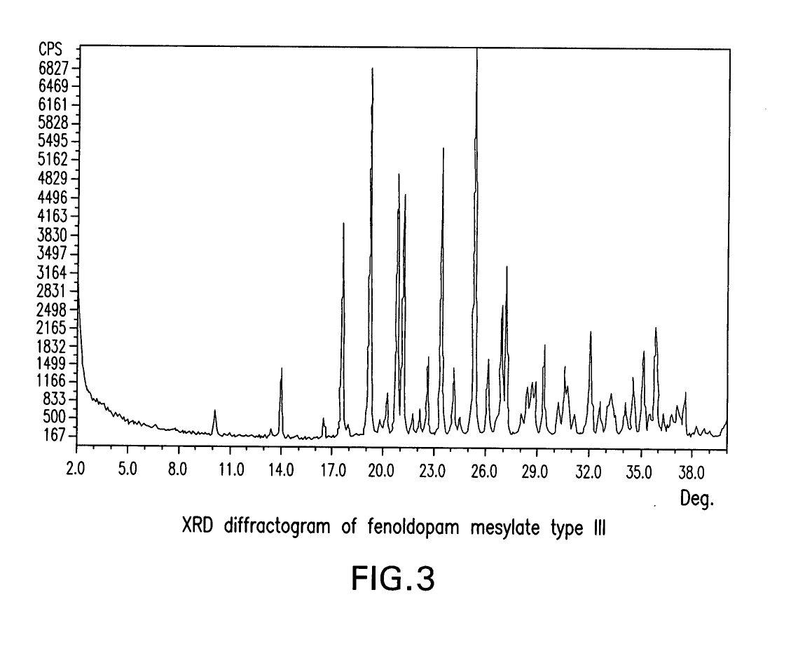

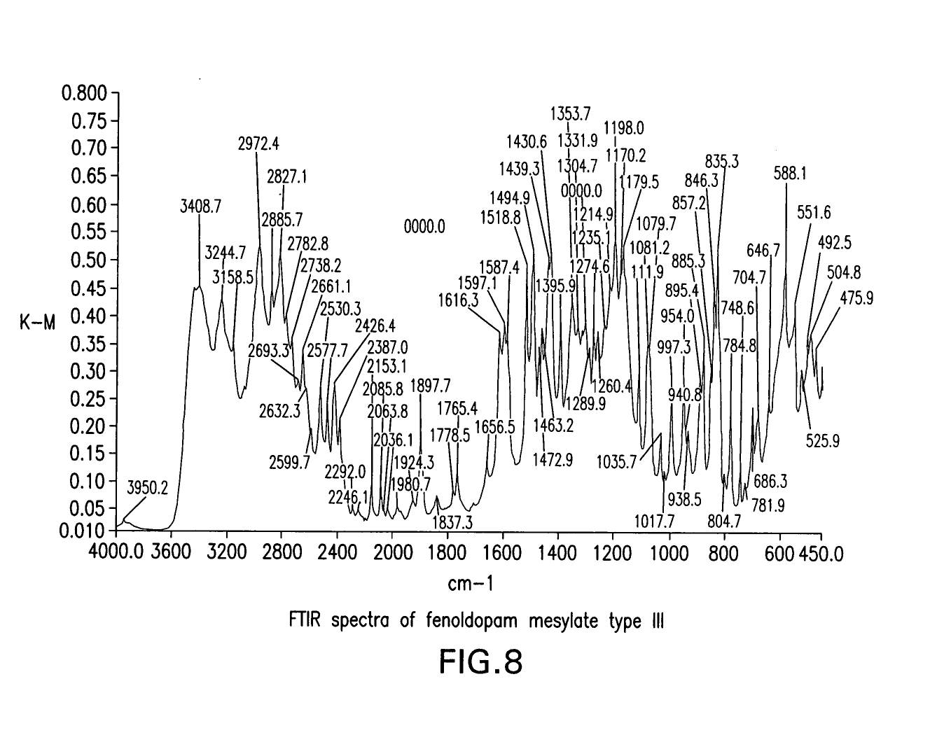

In a further embodiment, the invention is directed to a fenoldopam mesylate crystalline form characterized by data selected from the group consisting of a PXRD pattern with peaks at about 17.5°, 19.2°, 21.2°, 23.4°, and 25.3° 2θ±0.2° 2θ and a Fourier transform infrared spectroscopy (“FTIR”) spectrum with characteristic absorption bands in units of cm⁻¹ at about 588, 1170, 1198, 1439, and 1587 cm⁻¹. This fenoldopam mesylate crystalline is denominated herein as “Type III”. The Fenoldopam mesylate crystalline may be further characterized by data selected from the group consisting of a PXRD pattern with peaks at about 20.8°, 26.9°, 27.2°, 29.4°, and 32.1° 2θ±0.2° 2θ; FTIR absorption peaks at about 1431, 2827, 2972, 3245 and 3409 cm⁻¹; a PXRD pattern, substantially as depicted in FIG. 3; and an FTIR absorption spectrum substantially as depicted in FIG. 8. Preferably, the fenoldopam mesylate Type III corresponds to the anhydrous form, more preferably the fenoldopam mesylate is a non-hygroscopic anhydrous fenoldopam mesylate.

The present invention further provides substantially pure Fenoldopam mesylate Type III with less than about 5%, preferably, less than about 1%, of any other crystalline form of Fenoldopam mesylate, as measured by XRD, wherein any other crystalline form includes Type I, II, V, and VI.

Fenoldopam mesylate Type III may be prepared by providing a solution comprising fenoldopam mesylate and methanol; crystallizing fenoldopam mesylate Type III from the suspension; and recovering the crystals of fenoldopam mesylate Type III.

Preferably, the solution is acidified with methanesulfonic acid. Preferably, the solution is acidified using methanesulfonic acid to a pH of about 2 to about 4. Preferably, the acid is in an amount sufficient to provide the desired pH. Preferably, solvent is removed from the solution. Preferably, the solvent is removed by evaporation of the solvent to dryness. Preferably, the solution is triturated in boiling methanol. Preferably, crystallization of the solution is by admixing an anti-solvent or cooling the solution. Preferably, cooling the solution is preferably to a temperature of about 5° C. to about 0° C. Preferably, the anti-solvent is added in an amount sufficient to produce a suspension. Preferably, the anti-solvent is ethyl acetate. Most preferably, both cooling and anti-solvent are used to induce crystallization. Recovering the crystals is by any means known in the art, for example by filtration, washing, and drying. Preferably, washing is with the anti-solvent and ethyl ether in succession. Preferably, the crystals are dried under vacuum at a temperature of about 40° to about 80° C.

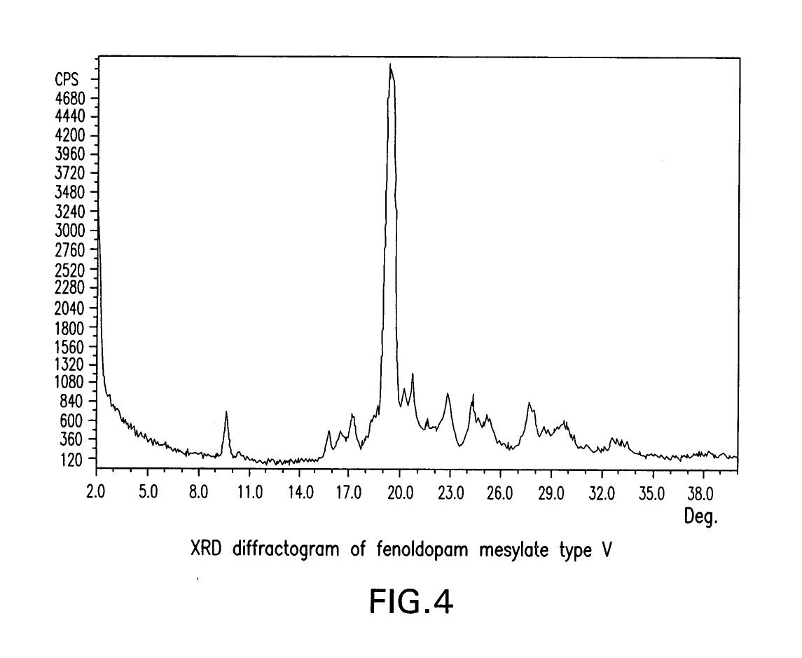

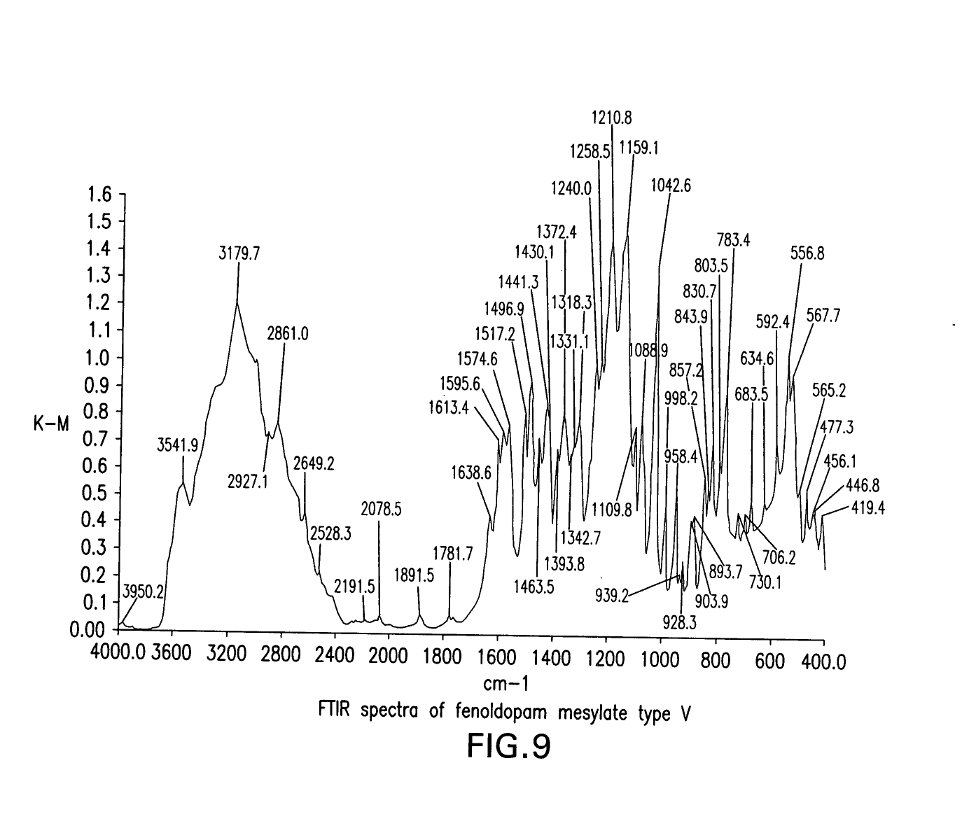

In a further embodiment, the invention is directed to a fenoldopam mesylate crystalline form characterized by data selected from the group consisting of a PXRD pattern with peaks at about 9.4°, 19.2°, 20.6°, 21.8°, and 25.3° 2θ±0.2° 2θ and a Fourier transform infrared spectroscopy (“FTIR”) spectrum with characteristic absorption bands in units of cm⁻¹ at about 1159, 1430, 1497, 1639, and 3542 cm⁻¹. This fenoldopam mesylate crystalline is denominated herein as “Type V”. The Fenoldopam mesylate crystalline may be further characterized by data selected from the group consisting of a PXRD pattern with peaks at about 15.8°, 16.5°, 17.2°, 20.3°, and 27.7° 2θ±0.2° 2θ; an FTIR absorption peaks at about 1043, 1211, 2528.3, 2649.2, 2927.1 cm⁻¹; a PXRD pattern, substantially as depicted in FIG. 4; and an FTIR absorption spectrum substantially as depicted in FIG. 9. The fenoldopam mesylate Type V is a hydrate form of fenoldopam mesylate, where the theoretical value of monohydrate is 4.3 percent by weight.

The present invention further provides substantially pure Fenoldopam mesylate Type V with less than about 10%, preferably, less than about 5%, more preferably, less than about 1%, of any other crystalline form of Fenoldopam mesylate, as measured by XRD, wherein any other crystalline form includes Form I, II, III, or VI. For example, substantially pure Fenoldopam mesylate Type V may contain less than about 5% of Types I, II, and III, and less than 10% of Type VI.

Fenoldopam mesylate Type V can be prepared by heating fenoldopam mesylate Type I. Preferably, fenoldopam mesylate Type I is heated to a temperature of about 80° to about 120° C. Preferably, heating is to a temperature of about 100° C. As one skilled in the art will appreciate, the time required to obtain crystalline fenoldopam mesylate will vary depending upon, among other factors, the amount of precipitate to be heated and the heating temperature, and can be determined by taking periodic PXRD readings.

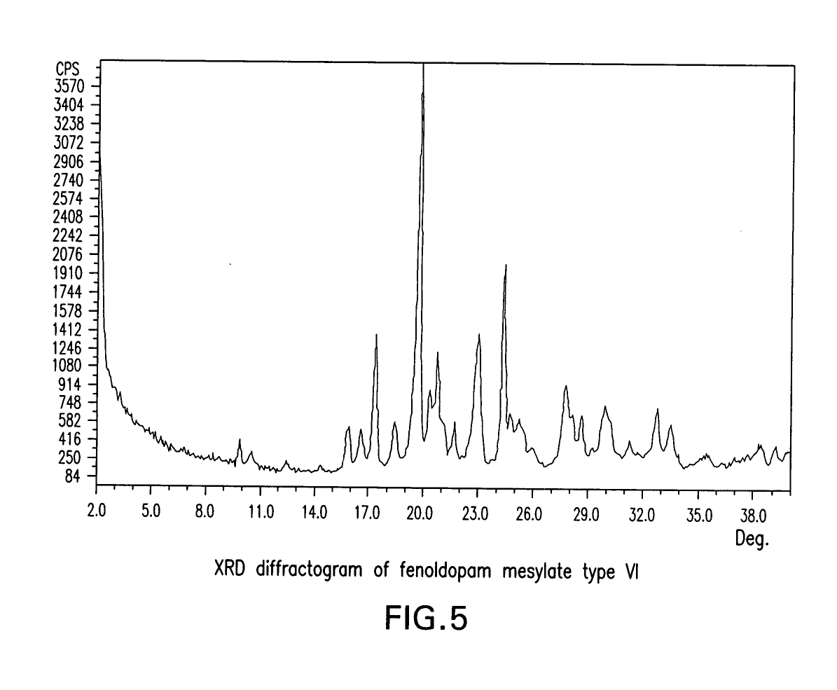

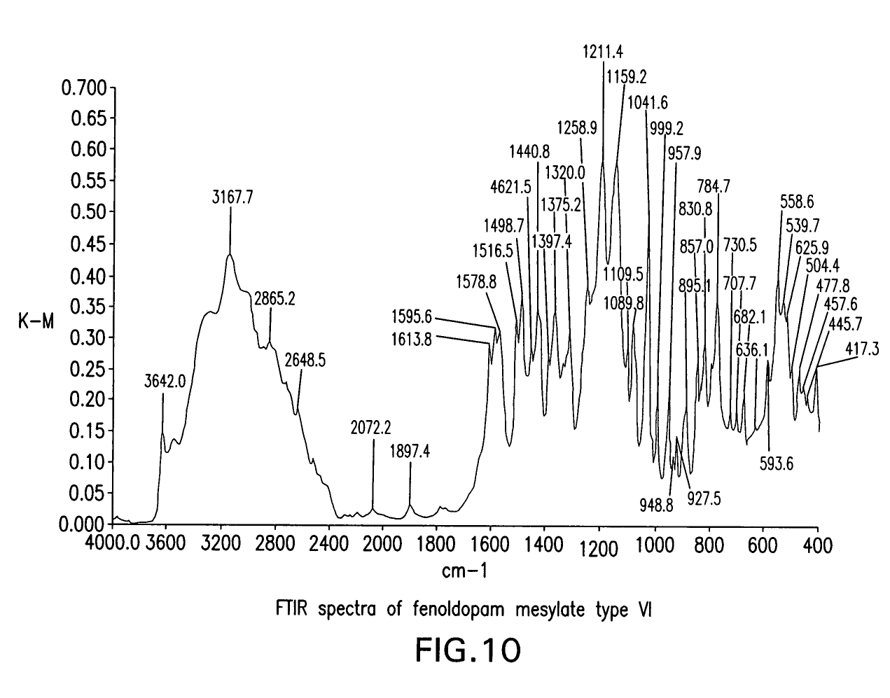

In a further embodiment, the invention is directed to a fenoldopam mesylate crystalline form characterized by data selected from the group consisting of a PXRD pattern having peaks at about 17.3°, 19.7°, 23.0°, 24.3°, and 30.0° 2θ±0.2° 2θ and a Fourier transform infrared spectroscopy (“FTIR”) spectrum with characteristic absorption bands in units of cm⁻¹ at about 559, 1259, 1579, 3168, and 3642 cm⁻¹. This fenoldopam mesylate crystalline is denominated herein as “Type VI”. The Fenoldopam mesylate crystalline may be further characterized by data selected from the group consisting of a PXRD pattern with peaks at about 15.8°, 16.6°, 20.3°, 27.8°, and 28.7° 2θ±0.2° 2θ; FTIR absorption peaks at about 785, 1320, 1376, 1463, and 2865 cm¹; a PXRD pattern, substantially as depicted in FIG. 5; and a FTIR absorption spectrum substantially as depicted in FIG. 10. The fenoldopam mesylate may have a TGA weight loss measured over the temperature range of 25° C. to 120° C. of about 4.6 to 5.3% by weight or a water content, as determined by Karl Fisher (KF), of about 4.6%. The fenoldopam mesylate may have a TGA weight loss measured over the temperature range of 25° C. to 100° C. of about 0.9% by weight. Both TGA and KF show the form to be a monohydrate or anhydrous, wherein the theoretical value for the monohydrate form is 4.3% by weight.

The present invention further provides substantially pure Fenoldopam mesylate Type VI with less than about 10%, preferably, less than about 5%, more preferably, less than about 1%, of any other crystalline form of Fenoldopam mesylate, as measured by XRD, wherein any other crystalline form includes Types I, II, III, and V. For example, substantially pure Fenoldopam mesylate Type VI may contain less than about 5% of Type I, II, and III, and less than 10% of Type V.

Fenoldopam mesylate Type VI can be prepared by applying solvent removal to Fenoldopam mesylate Type I. Solvent removal may be performed by exposing fenoldopam mesylate Type I to less than about 10% relative humidity for at least 5 days. Preferably, 8 days is sufficient. Preferably, fenoldopam mesylate Type I is exposed to 0 percent relative humidity. Alternatively, solvent removal may be performed by heating to a temperature of about 20° to about 70° C. Preferably, heating is under vacuum. Preferably, heating is to a temperature of about 40° C. As one skilled in the art will appreciate, the time required to obtain crystalline fenoldopam mesylate will vary depending upon, among other factors, the amount of precipitate and temperature to be exposed, and can be determined by taking periodic PXRD readings. Solvent removal results in Type VI having a TGA weight loss measured over the temperature range of 25° C. to 100° C. of about 0.9% by weight.

The size of fenoldopam mesylate crystalline forms Type I, Type III, Type V, and Type VI crystals is less than 300 μm.

In a further embodiment, the invention is directed to a process for the preparation of fenoldopam mesylate Type II, comprising providing a solution comprising isopropanol and fenoldopam mesylate; crystallizing to obtain fenoldopam mesylate; and recovering the fenoldopam mesylate Type II. Preferably, the solution is prepared by suspending fenoldopam mesylate Type I in isopropanol at a reflux temperature until dissolution. Preferably, crystallization is by cooling the solution to a temperature of about −20° C. to about 27° C. Recovery of the fenoldopam mesylate may be by any means known in the art such as by filtering, washing, and drying. Preferably, drying is at a temperature of about 60° C. to about 80° C. As one skilled in the art will appreciate, the time required to obtain crystalline fenoldopam mesylate will vary depending upon, among other factors, the amount of precipitate and temperature to of cooling, and can be determined by taking periodic PXRD readings

In a further embodiment, the invention is directed to a method of treatment of hypertension comprising administering a pharmaceutical composition comprising at least one of fenoldopam mesylate Types I, III, V, and VI to a patient in need thereof.

Pharmaceutical formulations of the present invention contain at least one of crystalline fenoldopam mesylate Types I, III, VI, V, and VI, optionally in mixture with other form(s) of fenoldopam mesylate. In addition to the active ingredient(s), the pharmaceutical formulations of the present invention may contain one or more excipients. Excipients are added to the formulation for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminum silicate, maltodextrin, methylcellulose, polymethacrylates, povidone (e.g., KOLLIDON® or PLASDONE®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL®, PRIMELLOSE®), colloidal silicon dioxide, croscarmellose sodium, crospovidone (e.g., KOLLIDON® or POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB®), and starch.

Glidants can be added to improve the flowability of a non-compacted solid composition, and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, fenoldopam mesylate and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer, such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

### EXAMPLES

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

X-Ray powder diffraction data were obtained by using methods known in the art with a SCINTAG powder X-Ray diffractometer model X'TRA equipped with a solid-state detector. The X-Ray radiation was copper radiation, having a wavelength of 1.5418 Å. A round aluminum sample holder with zero background was used for the measurements. All peak positions are within ±0.2° 2θ.

The TGA analysis was performed with a Mettler M3 thermogravimeter with samples of about 8 mg and a scan rate of 10° C./min from 25° C. to 200° C., subtracting a blank from the sample. The TGA oven was constantly purged with nitrogen gas at a flow rate of 40 ml/min, and standard 150 μl alumina crucibles covered by lids with 1 hole were used.

The FTIR spectroscopy measurements were performed with a Perkin-Elmer Spectrum One Spectrometer. The samples were analyzed using the diffuse reflectance technique (DRIFT). The samples were finely ground with potassium bromide, and the spectra were recorded using potassium bromide as the background in a diffused reflectance accessory. For each sample, 16 scans were performed over a range of 4000 to 400 cm⁻¹ with a resolution of 4.0 cm⁻¹.

The Karl Fisher analysis was performed with methods known art in the art with sample weights of at least 70 mg.

Melting points were measured in a Buchi instrument B-545, heating over a range of from 250° to 300° C. at 1.0 degree/min.

### Example 1

### Preparation of Fenoldopam Mesylate Type I

200 grams of fenoldopam mesylate were obtained and purified by column chromatography, followed by a single crystallization. The wet crystals of fenoldopam mesylate were then combined with a mixture of 560 grams of methanol and 1400 grams of water and 0.2 g of methanesulfonic acid, sufficient to provide a pH of 3.7 to form a solution, which was clarified through a disposable filter unit, and concentrated under vacuum in an evaporating flask with an external bath temperature of 60° C. to about 950 grams. The suspension was then cooled at 0° to 2° C. with stirring overnight, and then filtered. The filter cake was washed with 100 grams of cold water, and the product was dried in an oven at 80° C. under vacuum for 16 hours, providing a yield of 172.4 grams of product. An XRD analysis confirmed the sample was Type I.

### Example 2

### Preparation of Fenoldopam Mesylate Type I

200 grams of fenoldopam mesylate were obtained and purified by column chromatography, followed by a single crystallization. The wet crystals of fenoldopam mesylate were then combined with a mixture of 560 grams of methanol and 1400 grams of water to form a solution, which was clarified through a disposable filter unit, and concentrated under vacuum in an evaporating flask with an external bath temperature of 60° C. to about 950 grams. The suspension was then cooled at 0° to 2° C. with stirring for 2 hours, and then filtered. The filter cake was washed with 100 grams of cold water, and the product was dried in an oven at 80° C. under vacuum for 16 hours, providing a yield of product of 167.6 grams, containing 0.4 percent by weight water. An XRD analysis confirmed the sample was fenoldopam mesylate Type I.

### Example 3

### Preparation of Fenoldopam Mesylate Type I

165 grams of fenoldopam mesylate were obtained and purified by column chromatography, followed by a single crystallization. The wet crystals of fenoldopam mesylate were then combined with a mixture of 462 grams of methanol and 1155 grams of water to form a solution, which was clarified through a disposable filter unit, and concentrated under vacuum in an evaporating flask with an external bath temperature of 70° C. to about 770 grams. Then, 1000 grams of water were added, the suspension was evaporated to 770 grams, cooled at 0° to 2° C. with stirring for 2 hours, and then filtered. The filter cake was washed with 82 grams of cold water, and the product was dried in an oven at 80° C. under vacuum for 16 hours, providing a product yield of 101.8 grams, having an initial water content of 0.94 percent by weight. An XRD analysis confirmed the sample was fenoldopam mesylate Type I.

### Example 4

### Preparation of Fenoldopam Mesylate Type I

50 grams of fenoldopam mesylate were obtained and purified by column chromatography, followed by a single crystallization. The wet crystals of fenoldopam mesylate were then combined with a mixture of 140 grams of methanol and 350 grams of water to form a solution, which was clarified through a disposable filter unit, and concentrated under vacuum in an evaporating flask, having an external bath temperature of 70° C., to about 230 grams. The suspension was cooled at 0° to 2° C. with stirring for 2 hours, and then filtered. The filter cake was washed with 100 grams of cold water, and the product was dried in an oven at 80° C. under vacuum for 16 hours, providing a product yield of 38 grams, having an initial water content of 0.2 percent by weight. An XRD analysis confirmed the sample was fenoldopam mesylate Type I.

### Example 5

### Preparation of Fenoldopam Mesylate Type I

A 7 gram sample of fenoldopam mesylate was dissolved in 350 grams of water at 80° C. The solution was then cooled with stirring at room temperature, and the resulting suspension was left to crystallize in a refrigerator, without stirring, at a temperature of from 0° to 5° C. overnight. The suspension was then filtered. A small sample of the wet filter cake was analyzed by XRD, and determined to be fenoldopam mesylate Type I.

### Example 6

### Preparation of Fenoldopam Mesylate Type I

A 16 gram sample of fenoldopam mesylate was dissolved in 800 grams of water. The pH of the resulting solution was adjusted to 2.2 with methanesulfonic acid, the solution was concentrated under vacuum to 160 grams, and cooled with stirring at room temperature. The resulting suspension was then left to crystallize in a refrigerator, without stirring, at a temperature of from 0° to 5° overnight. The suspension was then filtered, and a small sample of this wet cake was analyzed by XRD, which demonstrated that the sample was fenoldopam mesylate Type I.

### Example 7

### Preparation of Fenoldopam Mesylate Type I

A production batch of 500 grams of fenoldopam hydrobromide was suspended in 5000 grams of methanol, under flushing nitrogen, and a solution of 113.6 grams of sodium bicarbonate in 2185 grams of water was added. After stirring for 30 minutes at room temperature, the suspension was filtered, and the product cake was washed with 2500 grams of water. The resulting 2687 grams of wet cake were suspended under nitrogen with 5000 grams of methanol, and 113.4 grams of methanesulfonic acid were added to adjust the pH of the solution to 2.5. The solution then was clarified through a disposable filter cartridge, rinsed with 600 grams of methanol, and concentrated in an evaporating flask under vacuum to a volume of about 3.8 liters. Then, 2500 grams of water were added, and the suspension was evaporated to 1.8 liters, and rotated slowly for 16 hours at room temperature. The precipitate was filtered, and washed with 250 grams of water. The mother liquor was concentrated to 0.8 liter, and left to crystallize for 16 hours at room temperature with slow rotation, providing a second crop of product, which was filtered, and then washed with 100 grams of isopropanol, and dried in a vacuum oven at 60° C. under vacuum for 16 hours, yielding 44 grams of gray material. An XRD analysis confirmed the sample was fenoldopam mesylate Type I.

### Example 8

### Preparation of Fenoldopam Mesylate Type I

A production batch of 500 grams of fenoldopam hydrobromide was suspended in 5000 grams of methanol under flushing nitrogen, and a solution of 113.6 grams of sodium bicarbonate in 2185 grams of water was added. After 30 minutes of stirring at room temperature, the suspension was filtered, and the product cake was washed with 2500 grams of water. The 2687 grams of wet cake was suspended under nitrogen with 5000 grams of methanol, and 113.4 grams of methanesulfonic acid were added to adjust the pH of the solution to 2.5. The solution was clarified through a disposable filter cartridge, rinsed with 600 grams of methanol, and concentrated in an evaporating flask under vacuum to a volume of about 3.8 liters. Then, 2500 grams of water were added, and the suspension was evaporated to 1.8 liters, and maintained with a slow rotation for 16 hours at room temperature. The resulting precipitate was filtered and washed with 250 grams of water. The mother liquor was concentrated to 0.8 liter, and left to crystallize for 16 hours at room temperature with slow rotation. The second crop product was filtered, washed with 100 grams of isopropanol, and then dried in a vacuum oven at 60° C. under vacuum for 16 hours. A total of 51 grams of a gray material was obtained. An XRD analysis confirmed the sample was fenoldopam mesylate Type I.

### Example 9

### Preparation of Type I from Type V

Exposure of a sample of fenoldopam mesylate Type V to 100 percent relative humidity for more than about 7 days at room temperature transforms this form to Type I, as determined by PXRD. TGA analysis determined that this form has a water content of 7.8 percent by weight water.

### Example 10

### TGA Analysis of Type I

When subjected to a thermogravimetric analysis (“TGA”), the measured TGA weight loss over a temperature range of from 25° to 130° C. is from about 5.5 to about 8.0 percent by weight. The TGA measurements provided results similar to the water content determined by Karl Fisher, demonstrating that fenoldopam mesylate Type I is a dihydrate, having a theoretical water content of 8.2 percent by weight.

### Example 11

### Stability Analysis Under Humid Conditions of Type I

Fenoldopam mesylate Type I was stored at 100 percent relative humidity for 8 days at room temperature. After exposure to these relative humidity conditions, PXRD analysis indicates that the sample remains fenoldopam mesylate Type I, as shown in Table 3.

TABLE 2
Initial form: Type I
Days: 8 days

| | Crystalline form |
|---|---|
| % RH | (by XRD) |
| Initial sample | Type I |
| 20 | Type I |
| 40 | Type I |
| 60 | Type I |
| 80 | Type I |
| 100 | Type I |

### Example 12

### Preparation of Fenoldopam Mesylate Type I

A solution of 300 grams of fenoldopam mesylate (after chromatography) in a mixture of 2420 grams of methanol and 17740 grams of water and 11 grams of methanesulfonic acid, sufficient to provide a pH of 2.3, was clarified through a disposable filter cartridge, rinsed with 200 grams of methanol, and concentrated under vacuum in an evaporating flask at an external bath temperature of 70° C. to about 1800 grams. The suspension was cooled at room temperature with stirring, left overnight, and then cooled at 0° to 2° C. for 2 hours, and filtered. The filter cake was washed with 210 grams of water, and the wet crystals were washed with 750 grams of isopropanol. An XRD analysis confirmed the sample was fenoldopam mesylate Type I.

### Example 13

### Preparation of Fenoldopam Mesylate Type I

A solution of 300 grams of fenoldopam mesylate (after chromatography) in a mixture of 2420 grams of methanol and 17740 grams of water and 11 grams of methanesulfonic acid, having a pH of 2.3, was clarified through a disposable filter cartridge, rinsed with 200 grams of methanol, and concentrated under vacuum in an evaporating flask at an external bath temperature of 70° C. to about 1800 grams. The suspension was cooled at room temperature with stirring, left overnight, then cooled at 0° to 2° for 2 hours, and filtered. The filter cake was washed with 210 grams of water, and an XRD analysis confirmed the sample was fenoldopam mesylate Type I.

### Example 14

### Preparation of Fenoldopam Mesylate Type II

The resulting filter cake of Example 12 was then suspended with 4800 grams of isopropanol in a flask, refluxed for half hour, and stirred for 2 hours at room temperature. The suspension was cooled at room temperature, filtered, and the crystals were rinsed with 210 grams of isopropanol. The product was dried in an oven at 80° C. under vacuum for 16 hours, providing 269 grams of product. An XRD analysis confirmed the sample was fenoldopam mesylate Type II.

### Example 15

### Preparation of Fenoldopam Mesylate Type II

The resulting filter cake in Example 13 was suspended with 4800 grams of isopropanol in a flask, refluxed for half hour, and stirred for 2 hours at room temperature. The suspension was cooled at room temperature, filtered, and the crystals were rinsed with 210 grams of isopropanol. The product was dried in an oven at 80° C. under vacuum for 16 hours, providing 260.5 grams of product. An XRD analysis confirmed the sample was fenoldopam mesylate Type II.

### Example 16

### Preparation of Fenoldopam Mesylate Type II

A solution of 100 grams of fenoldopam mesylate in a mixture of 840 grams of methanol and 6160 grams of water and an amount of methanesulfonic acid sufficient to provide a pH of 2.2 was concentrated under vacuum in an evaporating flask to 1200 grams. The suspension was cooled at room temperature with stirring, left overnight, then cooled at 0° to 2° C. for 2 hours, and filtered. The filter cake was washed first with 70 grams of water and then with 250 grams of isopropanol. The cake was suspended with 1600 grams of isopropanol, and refluxed for half hour in a flask. The suspension was cooled at room temperature, filtered, and the crystals were rinsed with 70 grams of isopropanol. The product was dried in an oven at 60° C. under vacuum for 16 hours, providing a yield of 88.6 grams of product having a water content of 0.22 percent by weight (0.1 percent by weight in a Solvias analysis). An XRD analysis confirmed the sample to be fenoldopam mesylate Type II.

### Example 17

### Preparation of Fenoldopam Mesylate Type II

A solution of 191 grams of fenoldopam mesylate in a mixture of 755 grams of methanol and 5860 grams of water and 0.2 grams of methanesulfonic acid, sufficient to provide a pH of 3.7, was clarified through a disposable filter cartridge, rinsed with 100 grams of water, and concentrated under vacuum in an evaporating flask at an external bath temperature of 70° C. to about 800 grams. Then, 1000 grams of water were added, and the suspension was evaporated to 0.8 liter. The suspension was cooled at room temperature with stirring, left overnight, and then cooled at 0° to 2° for 2 hours, and filtered. The filter cake was washed with 100 grams of water, suspended with 1900 grams of isopropanol in a flask, refluxed for a half hour, and stirred for 2 hours at room temperature. The suspension was cooled at room temperature, filtered, and the crystals were rinsed with a mixture of 490 grams of isopropanol and 10 grams of water. The product was dried in an oven at 80° under vacuum for 16 hours, providing a product yield of 176 grams, having a water content of 0.1 percent by weight. An XRD analysis confirmed the sample was fenoldopam mesylate Type II.

### Example 18

### Preparation of Fenoldopam Mesylate Type II

A solution of 45 grams of fenoldopam mesylate (after chromatography) in a mixture of 386 grams of methanol and 2830 grams of water, having a pH adjusted to 2.25 with 0.7 grams of methanesulfonic acid, was clarified through a disposable filter unit, and concentrated under vacuum in an evaporating flask at an external bath temperature of 60° C. to about 300 grams. The suspension was cooled with slow stirring at room temperature overnight, then at 0° to 2° for 2 hours, and then filtered. The filter cake was washed with 32 grams of cold water and then with 120 grams of isopropanol. The filter cake was suspended with 800 grams of isopropanol, and refluxed for half hour in a flask. The suspension was cooled at room temperature for 2 hours, filtered, and the crystals were rinsed with 32 grams of isopropanol. The product was dried in an oven at 60° C. under vacuum for 16 hours, providing a product yield of 40.4 grams, having a water content of 0.2 percent by weight. An XRD analysis confirmed the sample was fenoldopam mesylate Type II.

### Example 19

### Preparation of Fenoldopam Mesylate Type II

A solution of 100 grams of fenoldopam mesylate (after chromatography) in a mixture of 840 grams of methanol and 6160 grams of water, having a pH adjusted to 2.26, was clarified through a disposable filter unit, and concentrated under vacuum in an evaporating flask at an external bath temperature of 70° C. to about 1260 grams. The suspension was cooled with slow stirring at room temperature overnight, then at 0° to 2° C. for 2 hours, and then filtered. The filter cake was washed with 70 grams of cold water and then with 250 grams of isopropanol. The filter cake was suspended with 1600 grams of isopropanol, and refluxed for half hour in a flask. The suspension was cooled at room temperature for 2 hours, filtered, and the filtered crystals were rinsed with 70 grams of isopropanol. The product was dried in an oven at 60° C. under vacuum for 16 hours, providing a product yield of 91.5 grams, having a water content of 0.2 percent by weight, and an isopropanol content of 3112 ppm, based on the weight of the sample. An XRD analysis confirmed the sample was fenoldopam mesylate Type II

### Example 20

### Preparation of Fenoldopam Mesylate Type II

A solution of 190.6 grams of fenoldopam mesylate (after chromatography) in a mixture of 1680 grams of methanol and 12320 grams of water, having a pH adjusted to 2.25 with 1.4 grams of methanesulfonic acid, was clarified through a disposable filter unit, and concentrated under vacuum in an evaporating flask at an external bath temperature of 70° C. to about 1200 grams. The suspension was cooled with slow stirring at room temperature overnight, then at 0° to 2° for 2 hours, and then filtered. The filter cake was washed with 70 grams of cold water, and then with 250 grams of isopropanol. The filter cake was suspended in 1600 grams of isopropanol, and refluxed for half hour in a flask. The suspension was cooled at room temperature for 2 hours, filtered, and the resulting crystals were rinsed with 70 grams of isopropanol. The product was dried in an oven at 60° C. under vacuum for 16 hours, yielding 177.4 grams of product, having a water content of 0.2 percent by weight (0.08 percent by weigh by Solvias). An XRD analysis confirmed the product sample was fenoldopam mesylate Type II.

### Example 21

### Preparation of Fenoldopam Mesylate Type II

A solution of 197.5 grams of fenoldopam mesylate in a mixture of 780 grams of methanol and 5787 grams of water and 0.1 grams of methanesulfonic acid, sufficient to provide a pH of 3.66, was clarified through a disposable filter cartridge, rinsed with 100 grams of water, and concentrated under vacuum in an evaporating flask at an external bath temperature of 70° C. to about 800 grams. Then, 1000 grams of water were added, and the suspension was evaporated to 0.8 liter. The suspension was cooled at room temperature with stirring, left overnight, and then cooled at 0° to 2° C. for 2 hours, and filtered. The filter cake was washed with 100 grams of water, suspended with 1900 grams of isopropanol in a flask, refluxed for half hour, and stirred for 2 hours at room temperature. The suspension was cooled at room temperature, filtered, and the crystals were rinsed with a mixture of 490 grams of isopropanol and 10 grams of water. The product was dried in an oven at 80° under vacuum for 16 hours, providing a product yield of 179.4 grams, having a water content of 0.09 percent by weight. An XRD analysis confirmed the product sample was fenoldopam mesylate Type II.

### Example 22

### Fenoldopam Mesylate Type II—Stability

An 81.5 gram sample of the product produce in Example 17 was dried for 16 hours at 80° under vacuum, providing a yield of 81.2 grams, having a water content of 0.05 percent by weight, and an isopropanol content of 1620 ppm. An XRD analysis confirmed the product sample was fenoldopam mesylate Type II.

### Example 23

### Preparation of Fenoldopam Mesylate Type III

A 10 gram sample of fenoldopam mesylate was dissolved in 100 grams of methanol and 10 grams of water, than acidified with 0.4 grams (0.2 eq.) of methanesulfonic acid. The solution was evaporated to dryness at 75° C., the solid was triturated with 16 grams of boiling methanol for 30 minutes, and 40 grams of ethyl acetate were added. The suspension was cooled to 0° to 5° C. for 1 hour, filtered, and washed with 10 grams of ethyl acetate, then 10 grams of ethyl ether. The product was dried at 80° under vacuum for 16 hours, yielding 9.2 grams of product having a water content of 0.1 percent by weight. An XRD analysis confirmed the product sample was fenoldopam mesylate Type III.

### Example 24

### Preparation of Fenoldopam Mesylate Type III

A 10 gram sample of fenoldopam mesylate was dissolved in 400 grams of methanol, and the solution was evaporated to a volume of about 50 ml. The suspension was cooled to 0° to 5° C. for 2 hours, and filtered, providing 5.1 grams of product after drying at room temperature. An XRD analysis confirmed the product sample was fenoldopam mesylate Type III.

### Example 25

### Type III Stability—Heating

A 4.08 gram sample of the product of Example 22 was dried at 80° C. under vacuum for 16 hours, providing a yield of 3.95 grams, having a water content of 0.02 percent by weight and a methanol content of 766 ppm. An XRD analysis confirmed the product sample was fenoldopam mesylate Type III.

### Example 26

### Type III TGA Analysis

When subjected to a TGA, the measured TGA weight loss over the temperature range of from 25° to 100° C. is less than 0.1 percent by weight, indicating that fenoldopam mesylate Type III is anhydrous.

TABLE 3
Initial form: Anhydrous Type III
Days: 8 days

| | Water content | Crystalline form |
|---|---|---|
| % RH | (by TGA) | (by XRD) |
| 80 | — | Type III |
| 100 | 0.1% | Type III |
| Initial | <0.1% | Type III |
| sample | | |

### Example 27

### Preparation of Fenoldopam Mesylate Type V

About 100 mg of fenoldopam mesylate Type I, produced in Example 1, was heated to 100° C. for 30 minutes. The heated sample was analyzed by XRD, TGA, and FTIR, which confirmed the product was fenoldopam mesylate Type V.

### Example 28

### TGA Analysis of Type V

The measured TGA weight loss in the temperature range of 250 to 120° C. is about 5.6 percent by weight.

### Example 29

### Preparation of Fenoldopam Mesylate Type VI

Fenoldopam Type VI was produced when a mixture of about 50 mg of fenoldopam mesylate Type I, produced in Examples 2 and 4, was exposed to 0 percent relative humidity at room temperature for 8 days. The results of the XRD and TGA analysis of the exposed material are provided in Table 4.

TABLE 4
Hygroscopicity Results of Fenoldopam Mesylate Type I
Initial polymorphic form: Type I
Days: 8 days

| | Water content | Crystalline form |
|---|---|---|
| % RH | (by TGA) | (by XRD) |
| 0 | 0.9 | Type VI |
| 20 | 7.9 | Type I |
| 40 | 8.0 | Type I |
| 60 | 7.9 | Type I |
| 80 | 8.0 | Type I |
| Initial | 8.0 | Type I |

### Example 30

### Preparation of Fenoldopam Mesylate Type VI

The remaining part of the sample of fenoldopam mesylate Type I, produced in Example 5, was dried at 40° C. under vacuum for 16 hours, providing a product, having a water content of 1.19 percent by weight. Following an additional 16 hours of drying under the same conditions resulted in a water content of 2.71 percent by weight. When the product was left under a hood in ambient air for 3 days, 2.5 grams of product, having a water content of 4.7 percent by weight, was produced. An XRD analysis confirmed the product was fenoldopam mesylate Type VI.

### Example 31

### Preparation of Fenoldopam Mesylate Type VI

The remaining part of the sample produced in Example 6 was dried at 40° C. under vacuum for 16 hours, yielding a product, having a water content of 0.82 percent by weight. Following additional drying for 16 hours under those conditions, the water content was 0.94 percent by weight. Exposure to ambient air under a hood for 3 days resulted in a product having a water content of 4.63 percent by weight. An XRD analysis confirmed the product was fenoldopam mesylate Type VI.

### Example 32

### TGA Analysis of Fenoldopam Mesylate Type VI

When fenoldopam mesylate Type VI is subjected to TGA analysis, the TGA weight loss measured over the temperature range of 250 to 120° C. is about 4.6 to about 5.3 percent by weight, indicating that fenoldopam mesylate Type VI is hydrate form of fenoldopam mesylate, which has a theoretical value of 4.3 percent by weight, which corresponds to a monohydrate.

### Example 33

### Size of Crystals

The size of fenoldopam mesylate crystalline forms Type I, Type III, Type V, and Type VI crystals is less than 300 μm.

### Example 34

### Melting point

The melting point of fenoldopam mesylate Type I is about 262 to 264° C., the melting point of fenoldopam mesylate Type II is about 260 to 264° C., the melting point of fenoldopam mesylate Type III is about 256 to 271° C., the melting point of fenoldopam mesylate Type V is about 261 to 262° C., and the melting point of fenoldopam mesylate Type VI is about 261 to 264° C., as determined using a BUCHI melting point B-545 instrument, heating rate: 1.0°/minute.

While it is apparent that the invention disclosed herein is well calculated to fulfill the objects stated above, it will be appreciated that numerous modifications and embodiments may be devised by those skilled in the art. Therefore, it is intended that the appended claims cover all such modifications embodiments as falling within the true spirit and scope of the present invention.

What is claimed:

1. A fenoldopam mesylate hydrate.

2. A crystalline form Type I of fenoldopam mesylate, characterized by data selected from the group consisting of a PXRD pattern with peaks at about 16.4°, 18.8°, 21.8°, 23.9°, and 30.8° 2θ±0.2° 2θ and a Fourier transform infrared spectroscopy spectrum with characteristic absorption bands in units of cm⁻¹ at about 1210, 1571, 1643, 3178, and 3450 cm⁻¹.

3. The crystalline form of claim 2, further characterized by data selected from the group consisting of a PXRD pattern with peaks at about 8.8°, 9.4°, 15.8°, 20.3°, and 23.3° 2θ±0.2° 2θ; a PXRD pattern substantially as depicted in FIG. 1; an FTIR spectrum having absorption peaks at about 536, 546, 959, 1421, and 1442 cm⁻¹; an FTIR absorption spectrum substantially as depicted in FIG. 6; a TGA weight loss of about 5.5 to about 8.0% by weight over a temperature range of 250 to 130° C.; and crystal size of less than 300 μm.

4. The crystalline form of fenoldopam mesylate of claim 2, wherein the fenoldopam mesylate is a dihydrate.

5. The crystalline form of fenoldopam mesylate of claim 2, wherein the form is stable to exposure to 0 to 100% relative humidity conditions for more than 5 days.

6. The crystalline form of fenoldopam mesylate of claim 2, containing less than about 5% of any other crystalline form of Fenoldopam mesylate, as measured by XRD, wherein any other crystalline form includes Types II, III, V, and VI.

7. The crystalline form of fenoldopam mesylate of claim 6, containing less than about 1% of any other crystalline form of Fenoldopam mesylate, as measured by XRD, wherein any other crystalline form includes Type II, III, V, and VI.

8. A method of preparing the fenoldopam mesylate Type I of claim 2, providing a solution of fenoldopam mesylate and water, crystallizing fenoldopam mesylate from the solution; and recovering fenoldopam mesylate Type I.

9. The method of claim 8, wherein the solution of fenoldopam and water is prepared by heating a mixture of fenoldopam and water to a temperature of about 20° C. to about 100° C.

10. The method of claim 9, wherein heating is to a temperature of about 60° C. to about 80° C.

11. The method of claim 8, wherein methanesulfonic acid is admixed with the fenoldopam mesylate and water.

12. The method of claim 11, wherein methanesulfonic acid is admixed in an amount to provide a pH of about 2 to about 4.

13. The method of claim 11, wherein the mixture of fenoldopam and water further comprises methanol.

14. The method of claim 8, wherein crystallizing is initiated by concentrating the solution to form a suspension.

15. The method of claim 14, wherein concentrating the solution is by heating the solution to about 20° C. to about 90° C.

16. The method of claim 15, wherein heating is to a temperature of about 50° C. to about 90° C.

17. The method of claim 8, wherein crystallizing is by a process of cooling the solution to a temperature between 0° C. and 25° C.

18. The method of claim 17, wherein crystallizing is by a process of cooling the solution to a temperature between 0° C. and 10° C.

19. The method of claim 8, wherein recovery includes drying under vacuum at a temperature of about 500 to about 90° C.

20. A process for the preparation of the crystalline form Type I of fenoldopam mesylate of claim 2, comprising exposing fenoldopam mesylate Type V to more than 80% relative humidity for more than about 7 days.

21. The process in claim 20, wherein the relative humidity is about more than 90%

22. A crystalline form Type III of fenoldopam mesylate, characterized by data selected from the group consisting of a PXRD pattern with peaks at about 17.5°, 19.2°, 21.2°, 23.4°, and 25.3° 2θ±0.2° 2θ and a Fourier transform infrared spectroscopy spectrum with characteristic absorption bands in units of cm⁻¹ at about 588, 1170, 1198, 1439, and 1587 cm⁻¹.

23. The fenoldopam mesylate of claim 22, further characterized by data selected from the group consisting of a PXRD pattern with peaks at about 20.8°, 26.9°, 27.2°, 29.4°, and 32.1° 2θ±0.2° 2θ; an FTIR absorption peaks at 1431, 2827, 2972, 3245, and 3409 cm⁻¹; a PXRD pattern, substantially as depicted in FIG. 3; and an FTIR absorption spectrum substantially as depicted in FIG. 8.

24. The fenoldopam mesylate of claim 22, further characterized by a water content of less than about 0.1% by weight.

25. The fenoldopam mesylate of claim 24, which is a non-hygroscopic anhydrous fenoldopam mesylate.

26. The crystalline form of fenoldopam mesylate of claim 22, wherein the size of the crystals are less than 300 μm.

27. The crystalline form of fenoldopam mesylate of claim 22, comprising less than 5% by weight of any other crystalline form of Fenoldopam mesylate, as measured by XRD, wherein any other crystalline form includes Type I, II, V, and VI.

28. The crystalline form of fenoldopam mesylate of claim 27, comprising less than 1% by weight of any other crystalline form of Fenoldopam mesylate, as measured by XRD, wherein any other crystalline form includes Type I, II, V, and VI.

29. A method of preparing the fenoldopam mesylate Type III of claim 22, comprising providing a solution comprising fenoldopam mesylate and methanol; crystallizing fenoldopam mesylate Type III from the solution; and recovering the crystals of fenoldopam mesylate Type III.

30. The method of claim 29, wherein methanesulfonic acid is admixed with the fenoldopam mesylate and water.

31. The method of claim 30, wherein methanesulfonic acid is added in amount sufficient to bring the pH of the solution to between 2 and 4.

32. The method of claim 29, wherein the volume of the solution is reduced to obtain a reduced volume solution.

33. The method of claim 32, wherein the reduced volume solution is triturated in boiling methanol.

34. The method of claim 29, wherein crystallizing is initiated by cooling the solution.

35. The method of claim 29, wherein crystallizing is initiated by cooling to a temperature of 5° C. to about 0° C.

36. The method of claim 29, wherein crystallizing is initiated by admixing an anti-solvent.

37. The method of claim 36, wherein the anti-solvent is ethyl acetate.

38. The method of claim 29, wherein recovery is by drying under vacuum at a temperature of about 40° to about 80° C.

39. A fenoldopam mesylate crystalline form Type V, characterized by data selected from the group consisting of a PXRD pattern with peaks at about 9.4°, 19.2°, 20.6°, 21.8°, and 25.3° 2θ±0.2° 2θ and a Fourier transform infrared spectroscopy spectrum with characteristic absorption bands in units of cm⁻¹ at about 1159, 1430, 1497, 1639, and 3542 cm⁻¹.

40. The crystalline fenoldopam mesylate crystalline of claim 39, further characterized by data selected from the group consisting of a PXRD pattern with peaks at about 15.8°, 16.5°, 17.2°, 20.3°, and 27.7° 2θ±0.2° 2θ; an FTIR absorption peaks at 1043, 1211, 2528.3, 2649.2, 2927.1 cm⁻¹; a PXRD pattern, substantially as depicted in FIG. 4; and an FTIR absorption spectrum substantially as depicted in FIG. 9.

41. The crystalline fenoldopam mesylate crystalline of claim 39, having a water content of about 4.3% by weight which corresponds to the monohydrate form.

42. The crystalline form of fenoldopam mesylate of claim 39, wherein the size of the crystals is less than 300 μm.

43. The crystalline form of fenoldopam mesylate of claim 39, containing less than about 10% of any other form, as measured by XRD, wherein any other crystalline form includes Type I, II, III, and VI.

44. The crystalline form of fenoldopam mesylate of claim 43, containing less than about 5% of Type I, II, and III, and less than 10% of Type VI.

45. The crystalline form of fenoldopam mesylate of claim 43, comprising less than 5% by weight of any other form, as measured by XRD, wherein any other crystalline form includes Type I, II, III, and VI.

46. The crystalline form of fenoldopam mesylate of claim 45, comprising less than 1% by weight of any other form, as measured by XRD, wherein any other crystalline form includes Type I, II, III, and VI.

47. A method for preparing Fenoldopam mesylate Type V of claim 39, comprising heating fenoldopam mesylate Type I to obtain Fenoldopam mesylate Type V.

48. The method of claim 47, wherein heating is to a temperature of about 800 to about 120° C.

49. The method of claim 48, wherein heating is to about 100° C.

50. A crystalline form of fenoldopam mesylate Type VI, characterized by data selected from the group consisting of a PXRD pattern having peaks at 17.3°, 19.7°, 23.0°, 24.3°, and 30.0° 2θ±0.2° 2θ and a Fourier transform infrared spectroscopy spectrum with characteristic absorption bands in units of cm⁻¹ at about 559, 1259, 1579, 3168, and 3642 cm⁻¹.

51. The crystalline form of claim 50, further characterized by data by data selected from the group consisting of a PXRD pattern with peaks at about 15.8°, 16.6°, 20.3°, 27.8°, and 28.7° 2θ±0.2° 2θ; FTIR absorption peaks at about 785, 1320, 1376, 1463, and 2865 cm⁻¹; a PXRD pattern, substantially as depicted in FIG. 5; an FTIR absorption spectrum substantially as depicted in FIG. 10; a TGA weight loss measured over the temperature range of 25° C. to 120° C. of about 4.6 to 5.3% by weight; a TGA weight loss measured over the temperature range of 25° C. to 100° C. of about 0.9% by weight; and a water content, as determined by a Karl Fisher of about 4.6%.

52. The crystalline form of claim 50, which is a monohydrate or anhydrous form.

53. The crystalline form of fenoldopam mesylate of claim 50, wherein the size of the crystals are less than 300 μm.

54. The crystalline form of fenoldopam mesylate of claim 50, containing less than about 10% of any other form, as measured by XRD, wherein any other crystalline form includes Type I, II, III, and VI.

55. The crystalline form of fenoldopam mesylate of claim 50, containing less than about 5% of Type I, II, and III, and less than 10% of Type VI.

56. The crystalline form of fenoldopam mesylate of claim 50, comprising less than 5% by weight of any other form, as measured by XRD, wherein any other crystalline form includes Type I, II, III, and VI.

57. The crystalline form of fenoldopam mesylate of claim 56, comprising less than 1% by weight of any other form, as measured by XRD, wherein any other crystalline form includes Type I, II, III, and VI.

58. A method of preparing the fenoldopam mesylate Type VI of claim 50, from fenoldopam mesylate Type I, comprising solvent removal to obtain fenoldopam mesylate Type VI.

59. The method of claim 58, wherein solvent removal is by exposing fenoldopam mesylate Type I to less than about 10% relative humidity for at least 5 days.

60. The method of claim 59, wherein solvent removal is by exposing fenoldopam mesylate Type I to less than about 5% relative humidity.

61. The method of claim 60, wherein solvent removal is by exposing fenoldopam mesylate Type I to about 0 percent relative humidity at room temperature for at least 7 days.

62. The method of claim 58, wherein solvent removal is by heating to a temperature of about 20° to about 70° C.

63. The method of claim 62, wherein the temperature is about 40° C.

64. A pharmaceutical composition, comprising at least one crystalline form of fenoldopam mesylate selected from the group consisting of fenoldopam mesylate Type I, fenoldopam mesylate type III, fenoldopam mesylate type V, and fenoldopam mesylate type VI.

65. A method of preparing the pharmaceutical composition of claim 64, comprising blending at least one of the crystalline forms of fenoldopam mesylate and at least one pharmaceutical acceptable excipient.

66. A method of treatment of hypertension, comprising administering a pharmaceutical composition according to claim 64 to a patient in need thereof.
